(19) United States
(12) Patent Application Publication
(10) Pub. No.: US 2008/0210243 A1
(43) Pub. Date: Sep. 4, 2008

(54) Endotracheal cuff and technique for using the same

(76) Inventors: Jessica Clayton, San Jose, CA (US); Bryan Foronda, Stockton, CA (US); Ahmad Robert Hadba, Wallingford, CT (US)

Correspondence Address: NELLCOR PURITAN BENNETT LLC;ATTN: IP LEGAL, 60 Middletown Avenue, North Haven, CT, 06473, US

(21) Appl. No.: 11/713,551

(22) Filed: Mar. 2, 2007

(51) Int. Cl. A61M 16/04

(52) U.S. Cl. 128/207.15

(57) ABSTRACT

An inflatable balloon cuff associated with a tracheal tube may be adapted to reduce the passage of mucosal secretions into the lungs. A cuff as provided may include a sealing composition that plugs or seals any folds in the inflated cuff that may act as leak paths for mucosal secretions. Further, tracheal tube kits are provided for application and/or removal of the sealing composition.

### BACKGROUND OF THE INVENTION

1. Field of the Invention

The present invention relates to medical devices, and more particularly, to airway products, such as tracheal tubes and cuffs.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the course of treating a patient, a tube or other medical device may be used to control the flow of air, food, fluids, or other substances into and/or out of the patient. For example, medical devices such as tracheal tubes may be used to control the flow of one or more substances into or out of a patient. In many instances it is desirable to provide a seal between the outside of the tube or device and the interior of the passage in which the tube or device is inserted. In this way, substances can only flow through the passage via the tube or other medical device, allowing a medical practitioner to maintain control over the type and amount of substances flowing into and out of the patient.

For example, tracheal tubes may be used to control the flow of air or other gases through a patient's trachea. Such tracheal tubes may include endotracheal (ET) tubes or tracheostomy tubes. To seal these types of tracheal tubes, an inflatable cuff may be associated with these tubes. When inflated, the cuff generally expands into the surrounding trachea to seal the tracheal passage around the tube.

As many patients are intubated for several days, healthcare workers may need to balance achieving a high-quality tracheal seal with possible patient discomfort. Typical cuffs may be divided into low pressure cuffs and high pressure cuffs on the basis of their respective intracuff pressures after cuff inflation. High pressure cuffs are typically made of highly elastic materials that may form a relatively smooth seal against the trachea. However, these cuffs are associated with higher inflation pressures, as lower pressures are insufficient to overcome the natural initial resistance of the cuff material to stretching. Thus, high pressure cuffs are often inflated to at least twice the intracuff pressure of lower pressure cuffs. Because higher cuff pressures are associated with patient discomfort, physicians are often reluctant to inflate such high pressure cuffs fully in order to achieve an optimal seal. The mechanical pressure of the cuff against the tracheal walls may also cause temporary damage to cilial structures in the trachea that are associated with airway particle clearance. Thus, cilial injury may result in a temporary decrease in a patient's ability to remove bacteria or other foreign particles from the trachea.

While low pressure cuffs may be used to avoid patient discomfort, these low pressure cuffs may be associated with a lower quality cuff seal against the trachea. Although low pressure cuffs are generally made from more robust materials that are less elastic than high pressure cuffs, such cuffs may not achieve the smooth sealing surface associated with high pressure cuffs. For example, low cuff inflation pressures may be associated with allowing folds to form in the walls of the low pressure cuff that may serve as leak paths for air as well as microbe-laden secretions. In order to fit a range of trachea anatomies with a given size of tracheal tube, cuff diameters of low pressure cuffs are usually about one and a half times the diameter of the average trachea. Therefore, when inserted in an average-sized trachea, such a cuff is unable to fully expand and will fold in on itself within the trachea. These folds may serve as leak paths that allow microbe-laden secretions to flow past the cuff and enter the lung. Healthcare practitioners may attempt to overcome this problem by regularly aspirating any secretions that build up on the top surface of the cuff. However, such aspiration is time-consuming, and may not remove all of the mucosal secretions that have pooled on the top of the cuff.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

A tracheal tube kit is provided that includes: an inflatable balloon cuff including a distal opening and a proximal opening, wherein the distal opening and the proximal opening are suitably sized to accommodate a conduit; a conduit associated with the balloon cuff, wherein the conduit passes through the proximal opening and the distal opening of the balloon cuff; a lumen disposed on the conduit, wherein the lumen is adapted to apply a sealing composition to a surface of the balloon cuff; and a volume of sealing composition adapted to be operatively connected to the lumen, wherein the sealing composition includes a biocompatible viscous material.

A method of manufacturing a medical device is provided that includes: providing an inflatable balloon cuff including a distal opening and a proximal opening, wherein the distal opening and the proximal opening are suitably sized to accommodate a conduit; providing a conduit associated with the balloon cuff, wherein the conduit passes through the proximal opening and the distal opening of the balloon cuff; providing a lumen disposed on the conduit, wherein the lumen is adapted to apply a sealing composition to a surface of the balloon cuff; and providing a volume of sealing composition adapted to be operatively connected to the lumen, wherein the sealing composition includes a biocompatible viscous material.

A method of sealing a tracheal balloon is provided that includes: inflating a balloon cuff associated with a conduit in a patient's trachea, wherein the conduit passes through a proximal opening and a distal opening of the balloon cuff; applying a sealing composition to a surface of the inflated balloon cuff, wherein the sealing composition includes a biocompatible viscous material.

### BRIEF DESCRIPTION OF THE DRAWINGS

Advantages of the invention may become apparent upon reading the following detailed description and upon reference to the drawings in which:

FIG. 1 illustrates an endotracheal tube with an inflatable balloon cuff with a sealing composition in accordance with aspects of the present technique;

FIG. 2 illustrates the inflatable balloon cuff of the present techniques inserted into a patient's trachea; and

FIG. 3 illustrates an exemplary endotracheal tube kit including a syringe filled with a sealing composition that is adapted to be applied to an inflatable balloon cuff in accordance with aspects of the present technique.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In accordance with some aspects of the present technique, a tracheal tube with an inflatable cuff is provided that includes a sealing composition that is adapted to reduce or prevent the progress of mucosal secretions into the lungs. The sealing composition may be applied to an inflatable balloon cuff by a healthcare practitioner after the cuff has been inserted into a patient's trachea and inflated to an appropriate intracuff pressure. The sealing composition may be applied to the top surface of the inflated balloon cuff to seal any areas of the cuff that may not fit tightly against the tracheal walls. Further, the sealing composition may block the entry of liquids, such as mucosal secretions, into any folds in the cuff. Also provided herein are tracheal tube kits that include a suitable amount of a sealing composition to be applied to the top surface of the inflatable balloon cuff.

It is desirable to provide a medical balloon, such as an endotracheal cuff or other medical device that may substantially seal the passage in which the cuff is inserted so that mechanical ventilation can be used to introduce air, oxygen, or medications into the lungs. As cuffs are typically sized to be larger than the trachea when fully inflated in order to effectively seal a wide range of patient tracheas, the cuff walls are unable to inflate to their maximum diameter and may fold in on themselves, which may cause wrinkles and leak paths to form. The application of a sealing composition to the top surface of the balloon cuff may reduce or eliminate the ability of the mucosal secretions to flow through such leak paths. The sealing composition may effectively block the entrances of any leak paths in the seal of the cuff with the trachea such that the secretions may build up on the top of the sealing composition without flowing down through the wrinkles of the cuff.

The medical cuffs as provided herein may be used in conjunction with any suitable medical device. In certain embodiments, the cuffs as provided herein may be used in conjunction with a catheter, a stent, a feeding tube, an intravenous tube, an endotracheal tube, a tracheostomy tube, a circuit, an airway accessory, a connector, an adapter, a filter, a humidifier, a nebulizer, or a prosthetic, in various embodiments.

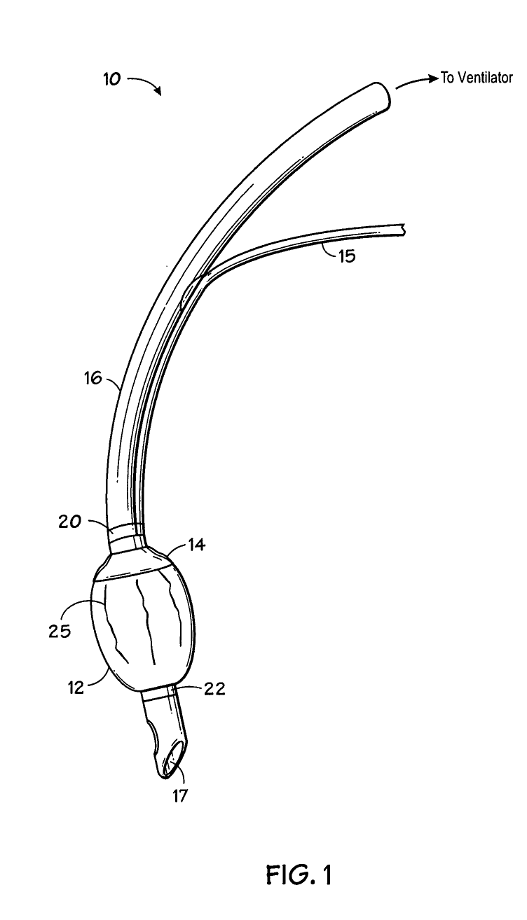

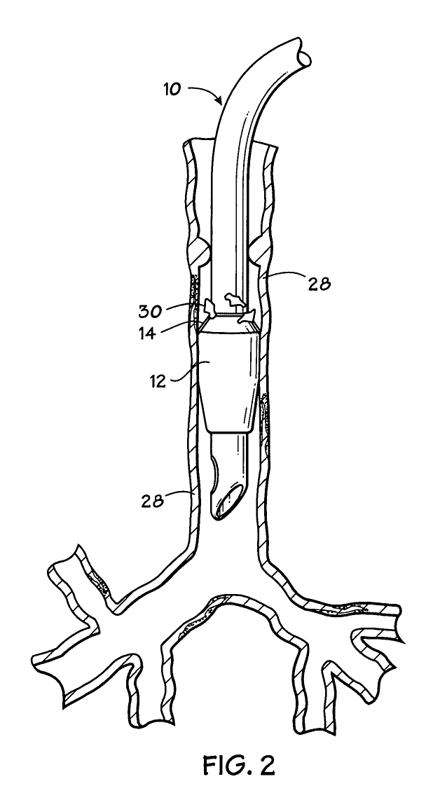

An example of a cuff used in conjunction with a medical device is a cuffed endotracheal tube 10, depicted in FIG. 1. The cuffed endotracheal tube 10 includes an inflatable cuff 12 that may be inflated to form a seal against the trachea wall 28 (see FIG. 2). In certain embodiments, the cuff 12 includes a sealing composition 14 that is disposed over the top of the cuff 12. The cuff is disposed on an endotracheal tube 16 that is suitably sized and shaped to be inserted into a patient and allow the passage of air through the endotracheal tube 16. Typically, the cuff is disposed, adhesively or otherwise, towards the distal end 17 of the endotracheal tube 16. The cuff 12 may be inflated and deflated via a lumen 15 in communication with the cuff 12, typically through a hole or notch in the lumen 15. The cuff 12 has a proximal opening 20 and a distal opening 22 formed in the cuff walls sized to accommodate the endotracheal tube 16. The proximal opening 20, located closer to the “machine end” of the tube 16, and a distal opening 22, located closer to the “patient end” of the tube 16, are typically used to mount the cuff 12 to the tube 16. When a cuff 12 is inflated into a patient's trachea, the area of the cuff 12 near the proximal opening 20 of the cuff walls may form a relatively flat surface that may tend to collect mucosal secretions. Although these secretions may be periodically aspirated, their collective pressure and weight between aspiration events may tend to accelerate the flow of these secretions down the leak paths created by the wrinkles 25. The sealing composition 14 may serve as a plug-like seal to prevent secretions from entering the wrinkles 25. The sealing composition 14 may be applied to the top of the cuff 12 prior to insertion of the tube 10 into the trachea. Alternatively, the sealing composition may be applied to the cuff 12 after insertion of the tube into the trachea.

The cuff 12 may be formed from materials having suitable mechanical properties (such as puncture resistance, pin hole resistance, tensile strength), chemical properties (such as forming a suitable bond to the tube 16), and biocompatibility. In one embodiment, the walls of the inflatable cuff 12 are made of a polyurethane having suitable mechanical and chemical properties. An example of a suitable polyurethane is Dow Pellethane® 2363-80A. In another embodiment, the walls of the inflatable cuff 12 are made of a suitable polyvinyl chloride (PVC). Other suitable materials include polypropylene, polyethylene teraphthalate (PET), low-density polyethylene (LDPE), silicone, neoprene, or polyisoprene.

The sealing composition 14 is configured to be disposed on the outer, tissue-contacting surface of the cuff 12 nearest to the proximal opening 20. FIG. 2 shows the exemplary cuffed endotracheal tube 10 inserted into a patient's trachea. As depicted, the sealing composition 14 may be applied to the cuff 12 so that it substantially covers the top surface of the cuff 12. The cuff 12 is inflated to form a seal against the tracheal walls 28. The sealing composition 14 is generally applied to the cuff 12 after the cuff 12 has been inflated. The sealing composition may be inserted into the patient's trachea from above the cuff 12 (e.g. from a syringe or other insertion device inserted into the mouth). Accordingly, the sealing composition may not coat the entire surface outer of the cuff 12 because much of the outer surface of the cuff 12 is in contact with the trachea or is generally not exposed. Therefore, the cuff surface on which the sealing composition 14 is applied may be a surface generally centered about the proximal opening 20 of the cuff 12 and may extend to the point at which the cuff 12 contacts the tracheal walls such that the sealing composition 14 forms a plug near the proximal opening 20. Mucosal secretions 30 may encounter the sealing composition 14 before they pass through the trachea into the lungs.

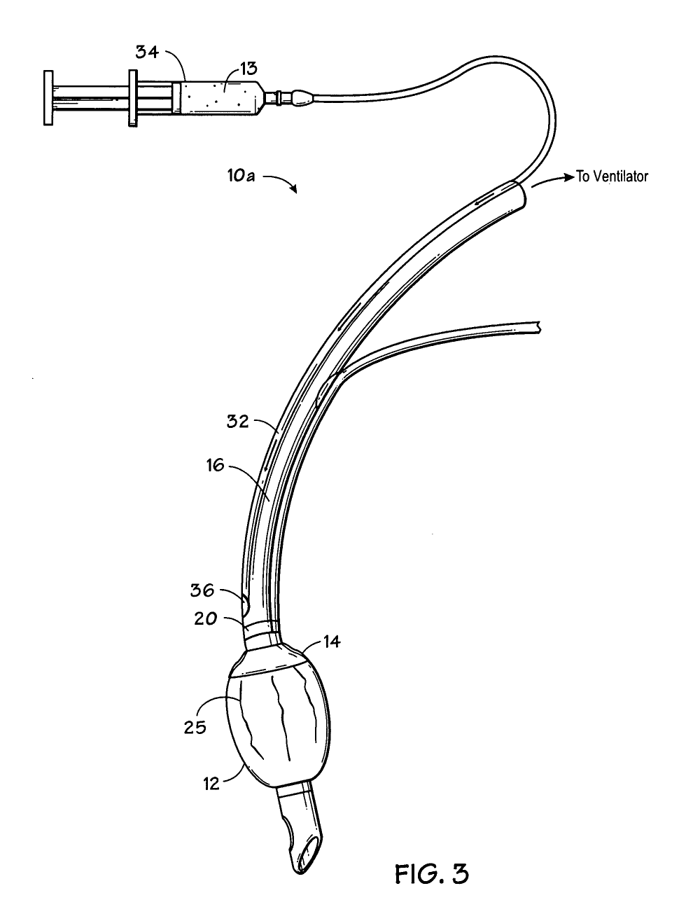

It is envisioned that the tracheal tubes as provided herein may be part of a tracheal tube kit that includes an appropriate dispensing device and an appropriate amount of a sealing composition 14. As depicted in FIG. 3, an endotracheal tube 10a may include a lumen 32 that is adapted to deliver the sealing composition 14 to the area of the cuff 12 near the proximal opening 20. The lumen 32 may be operatively connected to an appropriate dispensing device, such as a syringe 34, that may inject the sealing composition into the lumen 32. The lumen 32 may be disposed on the conduit 16, and may end in an opening 36 that is disposed directly above the proximal opening 20 of the cuff 12. In certain embodiments, the lumen 32 may also be configured to aspirate the sealing composition off the cuff 12 prior to removal of the endotracheal tube 10a from the trachea. Further, the lumen 32 may also be configured to aspirate off mucosal secretions that may have pooled at the top of the sealing composition 14. In such embodiments, the syringe 34 may be removed from the connection end of the lumen 32 so that an appropriate aspiration device (not shown) may be adapted to be operatively connected to the lumen 32. Further, the lumen 32 may also facilitate reapplication of the sealing composition 14 to the cuff 12 as necessary. In certain embodiments, the sealing composition may be reapplied at least every 24 hours, or at least every 48 hours. However, in other embodiments, the initial application of the sealing composition 14 may provide sufficient sealing of the cuff 12 for a week or more.

Generally, the dispensing device may be suitably sized and shaped to hold an appropriate amount of the sealing composition 14. In certain embodiments, the sealing composition 14 may be applied in volumes ranging from 1 mL-10 mL or more per application. Accordingly, a kit may include a prefilled syringe 34 containing an appropriate volume of sealing composition 14. The kit may also include any suitable number of prefilled syringes 34 for additional applications of the sealing composition 14 to the cuff 12. In a specific embodiment, an additional syringe 34 containing a volume of water, saline, or buffer may be include in the tracheal tube kit. Application of the contents of the additional syringe to the cuff 12 may loosen the sealing composition, which may facilitate extubation of the endotracheal tube 10a. Generally, any sealing composition 14 remaining in the trachea after extubation may be easily expelled by the body's natural expulsion mechanisms, such as the mucocilliary escalator and coughing.

In one embodiment, the syringe 34 and lumen 32 may deliver a precursor fluid 13 that may be processed after application to the cuff 12 in order to form the sealing composition 14 in situ. In such an embodiment, the precursor fluid 13 delivered by the lumen 32 may be substantially biocompatible. For example, an amide monomer solution may be delivered through the lumen 32 to the cuff 12 and cross-linked in place by adding a peroxide initiator. The precursor fluid 13 may be stored at room temperature in its liquid phase.

The sealing composition 14 may be any suitable biocompatible material that is sufficiently viscous to reduce or prevent the passage of mucosal secretions through wrinkles 25 in the cuff 12, but not so viscous as to be difficult to apply through a lumen and/or a syringe to a cuff 12. For example, the sealing composition may have a viscosity up to about 150,000 cP at 25° C. Materials having a viscosity substantially greater than 150,000 cP may be difficult to apply to the cuff 12. The sealing composition 14 may be sufficiently cross-linked to reduce its flowability so that it is not squeezed out of folds or tissue contact areas by pressures that are typical of cuff inflation pressures.

The sealing composition 14 may include gels, hydrogels, polymers, copolymer mixtures peptides, or polysaccharides. In certain embodiments, any gel or biocompatible polymer that is soluble in water and is of sufficient viscosity is appropriate for use as a sealing composition 14. In particular, the sealing composition 14 may include carboxymethyl cellulose, polyethylene glycol polymers, silicone gels, or other biocompatible gel-forming materials. For example, a 3-5% solution of carboxymethyl cellulose (average molecular weight 250,000) in water may be appropriate for use as a sealing composition 14. A 3% solution of high density carboxymethyl cellulose in water may have a viscosity ranging from about 2,000-17,000 cP. The viscosity of carboxymethyl cellulose may be influenced by its molecular weight and its degree of substitution.

The sealing composition may also include a polymer of N-isopropylacrylamide (N-IPAM). Such a polymer may form a photo-sensitive hydrogel as a copolymer of N-isopropylacrylamide and bis (4-(dimethylamino)phenyl) (4-vinylphenyl)methyl leucocyanide. Thus, the monomers may be initiated with the appropriate photoinitiation, which may be accomplished by exposing a precursor fluid 13 in the syringe 34 to light, or by shining light into the patient's trachea after the precursor fluid 13 has been applied to the cuff 12.

In one embodiment, the sealing composition includes hyaluronic acid, which is a naturally occurring linear polysaccharide composed of alternating disaccharide units of N-acetyl-D-glucosamine and D-glucuronidic acid. Hyaluronic acid is widely distributed in animal tissues, present in high concentrations in synovial fluid and the vitreous body of the eye, and in connective tissues of rooster comb, umbilical cord, and dermis. The molecular weight of hyaluronic acid isolated from natural sources generally falls within the range of about 6×10⁴ to about 1.2×10⁷ daltons. Naturally occurring hyaluronic acid does not have a strong foreign body reaction when implanted or injected into a living body and has excellent biocompatibility. The term hyaluronic acid may include any hyaluronate salts, including, sodium hyaluronate, potassium hyaluronate, magnesium hyaluronate, and calcium hyaluronate. The sealing composition 14 may include, for example, gels of hyaluronan (hyaluronic acid) cross-linked with vinyl sulfone or cross-linked mixtures of hyaluronan with other polymers or low molecular weight-substances

Biocompatibility of the sealing composition 14 may be enhanced by employing biodegradable molecules or polymers. For example, the sealing composition may include hydrolysable groups such as include polymers and oligomers of glycolide, lactide, epsilon-caprolactone, other hydroxy acids, and other biologically degradable polymers that yield materials that are non-toxic or present as normal metabolites in the body. Examples of such polymers include poly(alpha-hydroxy acids), poly(glycolic acid), poly(DL-lactic acid) and poly(L-lactic acid). Other useful materials include poly(amino acids), polycarbonates, poly(anhydrides), poly(orthoesters), poly(phosphazines) and poly(phosphoesters). Polylactones such as poly(epsilon-caprolactone), poly(delta-caprolactone), poly(delta-valerolactone) and poly(gamma-butyrolactone), for example, are also useful.

Alternatively, the sealing composition 14 may include a thermoreversible gel such as polymers composed of polyoxypropylene and/or polyoxyethylene. These polymers have the ability to change from the liquid state to the gel state at temperatures close to body temperature. The liquid state-to-gel phase transition is dependent on the polymer concentration and the ingredients incorporated into the solution. Accordingly, the concentration of the polymer may be adjusted in order to obtain a phase transition temperature of, for example, 28° C.-37° C. More specifically, the sealing composition may include Pluronics®, a family of poly(ethylene oxide)-poly(propylene oxide)-poly(ethylene oxide) (PEO-PPO-PEO) triblock copolymers that exhibit low toxicity and minimal immune response. Certain poloxamers are useful in providing additional benefits, such as in maintaining gel viscosity. Poloxamers are ABA tri-block co-polymers consisting of polyethylene oxide (PEO) and polypropylene oxide (PPO), and have the general formula HO(CH₂CH₂O)a((CH₃)CHCH₂O) b(CH₂CH₂O)ₐ—H in which “a” is generally from 2 to 130 and “b” is generally from 15 to 67, although it will be appreciated that other values for “a” and “b” are also possible. Poloxamers are amphipathic in nature due to the relative hydrophobicity of the central (PO) core and hydrophilicity of the EO end blocks. They are commercially available in varying compositions under the generic name poloxamers [trade names Pluronics® (BASF) and Synperonics® (ICI)]. The term poloxamer generally applies to any block copolymer of ethylene oxide and propylene oxide which is suitable for use in the present invention, and wherein each “a” may be the same or different.

Optionally, therapeutically beneficial compounds may be incorporated into the water-swellable layer 14. The biologically-active agent may be soluble in the polymer solution to form a homogeneous mixture, or insoluble in the polymer solution to form a suspension or dispersion. Over time, the biologically-active agent may be released from the cuff 12 into the adjacent tissue fluids, for example at a controlled rate. The release of the biologically-active agent from the present composition may be varied, for example, by the solubility of the biologically-active agent in an aqueous medium, the distribution of the agent within the composition, ion exchange, pH of the medium, the size, shape, porosity, solubility and biodegradability of the article or coating, and the like. The term “therapeutically beneficial compound” encompasses therapeutic agents, such as drugs, and also genetic materials and biological materials.

A variety of therapeutically beneficial compounds may be included, such as those detailed in International Patent Application WO200623486 by Hadba et al, which is hereby incorporated by reference in its entirety herein. For example, the therapeutically beneficial compound may include proteins (including enzymes, growth factors, hormones and antibodies), peptides, organic synthetic molecules, inorganic-compounds, natural extracts, nucleic acids (including genes, telomerase inhibitor genes, antisense nucleotides, ribozymes and triplex forming agents), lipids and steroids, carbohydrates (including heparin), glycoproteins, polymeric drugs, e.g. polysalicilic acid, prodrugs, and combinations thereof. The therapeutically beneficial compound may have a variety of biological activities, such as vasoactive agents, neuroactive agents, hormones, anticoagulants, immunomodulating agents, cytotoxic agents, antibiotics, antivirals, or may have specific binding properties such as antisense nucleic acids, antigens, antibodies, antibody fragments or a receptor. Proteins including antibodies or antigens can also be delivered. Proteins are defined as consisting of 100 amino acid residues or more; peptides are less than 100 amino acid residues. Unless otherwise stated, the term protein refers to both proteins and peptides. Examples include insulin and other hormones.

The tracheal cuffs of the present techniques may be incorporated into systems that facilitate positive pressure ventilation of a patient, such as a ventilator. Such systems may typically include connective tubing, a gas source, a monitor, and/or a controller. The controller may be a digital controller, a computer, an electromechanical programmable controller, or any other control system.

Typically, endotracheal cuffs are inflated within a patient's trachea such that the intra cuff pressure is approximately 20-25 cm H₂O. Endotracheal cuffs utilizing inflation pressures significantly greater 50 cm H₂O may be referred to as high-pressure cuffs, while cuffs that are able to effectively seal the trachea at pressures less than 30 cm H₂O may be considered low-pressure cuffs. In certain embodiments, intra cuff inflation pressures of 10-30 cm H₂O may be used with the cuffs of the present techniques.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. A tracheal tube kit comprising:
an inflatable balloon cuff comprising a distal opening and a proximal opening, wherein the distal opening and the proximal opening are suitably sized to accommodate a conduit;
a conduit associated with the balloon cuff, wherein the conduit passes through the proximal opening and the distal opening of the balloon cuff,
a lumen disposed on the conduit, wherein the lumen is adapted to apply a sealing composition to a surface of the balloon cuff; and
a volume of sealing composition adapted to be operatively connected to the lumen, wherein the sealing composition comprises a biocompatible viscous material.

2. The tracheal tube kit, as set forth in claim 1, wherein the balloon cuff comprises polyethylene teraphthalate (PET), low-density polyethylene (LDPE), polyvinyl chloride (PVC), silicone, neoprene, polyisoprene, or polyurethane (PU).

3. The tracheal tube kit, as set forth in claim 1, wherein the sealing composition comprises hyaluronic acid.

4. The tracheal tube kit, as set forth in claim 1, wherein the sealing composition comprises carboxymethyl cellulose.

5. The tracheal tube kit, as set forth in claim 1, wherein the sealing composition comprises a precursor fluid that is adapted to increase in viscosity upon application of heat or light.

6. The tracheal tube kit, as set forth in claim 1, wherein the sealing composition comprises a hydrogel.

7. The tracheal tube kit, as set forth in claim 1, wherein the sealing composition comprises a polymer.

8. The tracheal tube kit, as set forth in claim 1, wherein the sealing composition comprises a thermoreversible compound.

9. The tracheal tube kit, as set forth in claim 1, wherein the sealing composition is cross-linked.

10. The tracheal tube kit, as set forth in claim 1, wherein the sealing composition has a viscosity greater than 500 cp.

11. The tracheal tube kit, as set forth in claim 1, wherein the sealing composition comprises a therapeutically beneficial compound.

12. The tracheal tube kit, as set forth in claim 1, comprising a syringe adapted to hold the volume of sealing composition.

13. A method of manufacturing a medical device, comprising:
providing an inflatable balloon cuff comprising a distal opening and a proximal opening, wherein the distal opening and the proximal opening are suitably sized to accommodate a conduit;
providing a conduit associated with the balloon cuff, wherein the conduit passes through the proximal opening and the distal opening of the balloon cuff,
providing a lumen disposed on the conduit, wherein the lumen is adapted to apply a sealing composition to a surface of the balloon cuff; and
providing a volume of sealing composition adapted to be operatively connected to the lumen, wherein the sealing composition comprises a biocompatible viscous material.

14. The method, as set forth in claim 13, wherein the providing the balloon cuff comprises providing a balloon cuff comprising polyethylene teraphthalate (PET), low-density polyethylene (LDPE), polyvinyl chloride (PVC), silicone, neoprene, polyisoprene, or polyurethane (PU).

15. The method, as set forth in claim 13, wherein providing the sealing composition comprises providing hyaluronic acid.

16. The method, as set forth in claim 13, wherein providing the sealing composition comprises providing carboxymethyl cellulose.

17. The method, as set forth in claim 13, wherein providing the sealing composition comprises providing a precursor fluid that is adapted to increase in viscosity upon application of heat or light.

18. The method, as set forth in claim 13, wherein providing the sealing composition comprises providing a hydrogel.

19. The method, as set forth in claim 13, wherein providing the sealing composition comprises providing a polymer.

20. The method, as set forth in claim 13, wherein providing the sealing composition comprises providing a thermoreversible compound.

21. The method, as set forth in claim 13, wherein providing the sealing composition comprises providing a cross-linked composition.

22. The method, as set forth in claim 13, wherein providing the sealing composition comprises providing a composition having a viscosity greater than 500 cp.

23. The method, as set forth in claim 13, comprising providing a therapeutically beneficial compound in the sealing composition.

24. The method, as set forth in claim 13, comprising providing a syringe adapted to hold the volume of sealing composition.

25. A method of sealing a tracheal balloon, comprising:
inflating a balloon cuff associated with a conduit in a patient's trachea, wherein the conduit passes through a proximal opening and a distal opening of the balloon cuff;
applying a sealing composition to a surface of the inflated balloon cuff, wherein the sealing composition comprises a biocompatible viscous material.

26. The method, as set forth in claim 25, comprising applying heat or light to the sealing composition to increase its viscosity.

27. The method, as set forth in claim 25, comprising reapplying the sealing composition to the surface of the inflatable balloon cuff at least every 24 hours.

28. The method, as set forth in claim 25, wherein applying the sealing composition comprises injecting the sealing composition into a lumen adapted to deliver the sealing composition to the surface of the inflatable balloon cuff.

29. The method, as set forth in claim 25, comprising applying a solution to the surface of the inflatable balloon cuff that is adapted to loosen or dilute the sealing composition prior to removal of the balloon cuff from the patient's trachea.

30. The method, as set forth in claim 25, comprising aspirating the sealing composition from the surface of the inflatable balloon cuff prior to removal of the balloon cuff from the patient's trachea.
